(19) United States
(12) Patent Application Publication
(10) Pub. No.: US 2025/0312002 A1
(43) Pub. Date: Oct. 9, 2025

(54) INTRACARDIAC ECHOCARDIOGRAPHY (ICE) CATHETER HANDLE STABILIZER

(71) Applicant: KONINKLIJKE PHILIPS N.V., EINDHOVEN (NL)

(72) Inventors: Jacob ROUW, SAN DIEGO, CA (US); Drew HOFFMAN, SAN DIEGO, CA (US)

(21) Appl. No.: 19/097,186

(22) Filed: Apr. 1, 2025

Related U.S. Application Data
(60) Provisional application No. 63/575,945, filed on Apr. 8, 2024.

(51) Int. Cl. A61B 8/00; A61B 8/12; A61B 50/20

(52) U.S. Cl. CPC A61B 8/4209; A61B 8/12; A61B 50/20; A61B 2560/0462

(57) ABSTRACT

A method of intraluminal imaging may comprise inserting an intraluminal imaging device into a patient body, advancing a distal portion of the intraluminal imaging device to a desired location within the patient body, coupling a handle of the intraluminal imaging device to an intraluminal imaging device stabilizer, and obtaining imaging data of a region of interest of the patient body. An intraluminal imaging system may comprise an intraluminal imaging device including a handle and a distal portion sized and shaped for introduction into a patient body. The intraluminal imaging system may also comprise an intraluminal imaging device stabilizer including a base component and an engagement component coupled to the base component. The engagement component may include a raised support structure sized and shaped for snap-fit engagement with the handle of the intraluminal imaging device. Associated devices, systems, and methods are also provided.

### FIELD

The present disclosure relates generally to stabilizers for intraluminal medical imaging systems and associated workflows. In some aspects, intracardiac echocardiography (ICE) catheter handle stabilizers and associated workflows are provided.

### BACKGROUND

Diagnostic and therapeutic ultrasound catheters have been designed for use inside many areas of the human body. In the cardiovascular system, two common diagnostic ultrasound methods are intravascular ultrasound (IVUS) and intra-cardiac echocardiography (ICE). In some implementations, a single rotating transducer or an array of transducer elements is used to transmit ultrasound at the distal portion of the imaging catheters. The same transducers and/or separate transducers may be used to receive echoes from the tissue. A signal generated from the echoes is transferred to a console which allows for the processing, storing, displaying, and/or manipulating the ultrasound-related data.

IVUS catheters are often used in the large and small blood vessels (arteries or veins) of the body and are almost always delivered over a guidewire having a flexible tip. ICE catheters are often used to image chambers of the heart and surrounding structures, for example, to guide and facilitate medical procedures, such as transseptal lumen punctures, left atrial appendage closures, atrial fibrillation ablation, and valve repairs. Commercially available ICE catheters are typically not designed to be delivered over a guidewire, but instead have distal ends that can be articulated by a steering mechanism located in a handle at the proximal end of the catheter. For example, an ICE catheter may be inserted through the femoral or jugular artery when accessing the anatomy and steered in the heart to acquire images beneficial for ensuring the safety of the associated medical procedures.

ICE catheters, like many other intraluminal imaging catheters, are typically controlled by an operator at the operating table on which the patient is positioned. The ICE catheter may be inserted into a lumen of the patient, such as a blood vessel, and an imaging tip of the catheter may be navigated through the vasculature to a desired location to image a region of interest. The ICE catheter may be navigated by maneuvering a handle attached to the ICE catheter and/or by manipulating one or more movement controls disposed on the handle. This process can take time, as the operator (e.g., physician) must orient themselves in complex anatomy and make medical decisions surrounding diagnosis and/or treatment. In some instances, the position of an imaging face or transducer array of the ICE catheter may be changed by small rotational and/or translational movement of the ICE catheter handle. Therefore, it can be important to maintain stability of the ICE catheter handle throughout the procedure, especially when obtaining ultrasound data.

When the imaging tip has reached the desired imaging location, the operator may continue to hold the handle during the procedure or ask another person to hold the handle to avoid accidentally moving the imaging tip from the desired location and/or unwantedly triggering one of the movement controls on the handle. Further, placing the handle on the operating table, patient, and/or other surface can also result in undesirable movement of the imaging tip inside the patient. For example, in some instances, the handle may be ergonomically designed to be held in a hand (e.g., having a vaguely cylindrical shape). As a result, when the ergonomically designed handle is set down on a surface (e.g., operating table or bed) it may roll over or tilt in an unwanted manner, potentially altering the location and/or orientation of the imaging tip. Additionally, if an assistant is holding the device for the physician, the assistant may be prevented from helping with other tasks and/or handling other responsibilities associated with the procedure. Further, it is often not practical or efficient for the primary operator to continuously hold the handle and/or require another person to scrub in to hold the handle.

### SUMMARY

The present disclosure advantageously describes intraluminal medical imaging interface devices and systems that can improve the workflow of an intraluminal medical imaging procedure. While existing intraluminal imaging devices and associated systems and workflows have proved useful, there remains a need for improved devices, systems, and methods. Aspects of the present disclosure provide such improved devices, systems, and methods.

In some aspects, an intra-cardiac echocardiography (ICE) catheter handle stabilizer is provided as a disposable plastic sterilized holder shipped in two pieces. The two pieces of the stabilizer may be press fit together. The stabilizer may be used on a procedure table during an ICE imaging procedure used to secure the ICE catheter handle in position and enable controlled clocking motions of the ICE Catheter, avoiding unwanted movement of the ICE catheter and/or avoiding the need for another person to hold the ICE catheter while the primary operator attends to other tasks. In some aspects, the stabilizer may also secure the handle of the ICE catheter in position within sterile packaging during shipping and/or transport to the procedure room.

In some aspects, a method of intraluminal imaging comprises: inserting an intraluminal imaging device into a patient body; advancing a distal portion of the intraluminal imaging device to a desired location within the patient body; coupling a handle of the intraluminal imaging device to an intraluminal imaging device stabilizer; and obtaining imaging data of a region of interest of the patient body using the intraluminal imaging device. Coupling the handle of the intraluminal imaging device to the intraluminal imaging device stabilizer may include snap-fitting the handle to a raised support structure of the intraluminal imaging device stabilizer. In some instances, the method includes rotating, while the handle of the intraluminal imaging device is positioned within the raised support structure of the intraluminal imaging device stabilizer, the handle of the intraluminal imaging device relative to the intraluminal imaging device stabilizer. Rotating the handle may adjust an orientation of the distal portion of the intraluminal imaging device with the patient body. The method may include moving, while the handle of the intraluminal imaging device is coupled to the intraluminal imaging device stabilizer, the handle of the intraluminal imaging device and the intraluminal imaging device stabilizer to adjust a position of the distal portion of the intraluminal imaging device. Further, the method may include adjusting, while the handle of the intraluminal imaging device is coupled to the intraluminal imaging device stabilizer, an orientation of an imaging array in the distal portion of the intraluminal imaging device using at least one actuator on the handle of the intraluminal imaging device. In some aspects, the method may include assembling the intraluminal imaging device stabilizer. Assembling the intraluminal imaging device stabilizer may include coupling a base component and an engagement component. The engagement component may include the raised support structure. Coupling the base component and the engagement component may include press fitting the base component and the engagement component together.

In some aspects, an intraluminal imaging system comprises: an intraluminal imaging device including a handle sized and shaped for handheld use; and a distal portion coupled to the handle, the distal portion sized and shaped for introduction into a patient body and including an imaging array; and an intraluminal imaging device stabilizer including a base component; and an engagement component coupled to the base component. The engagement component may include a raised support structure sized and shaped for snap-fit engagement with the handle of the intraluminal imaging device. The base component may be press fit engaged with the engagement component. The engagement component may extend across the base component in a direction perpendicular to a length of the base component. In some aspects, the base component and the engagement component each have a rectangular outer profile when viewed from above such that the base component and the engagement component form an X or cross shape when press fit together and viewed from above. The raised support structure may include a first support arm, a second support arm, and a cradle extending between and connecting the first support arm and the second support arm. The cradle may be sized and shaped for snap-fit engagement with the handle of the intraluminal imaging device. In some aspects, each of the first support arm and the second support arm extend at an oblique angle with respect to a base plate of the engagement component. The first and second support arms may be sized and shaped to maintain the handle of the intraluminal imaging device in a raised position spaced from a surface on which the intraluminal imaging device stabilizer is positioned when the handle of the intraluminal imaging device is snap-fit engaged with the cradle.

In some aspects, a stabilizer for an intraluminal imaging device that comprises: a base component; and an engagement component coupled to the base component, the engagement component including a raised support structure sized and shaped for snap-fit engagement with a handle of an intraluminal imaging device. The engagement component comprises a first support arm, a second support arm, and a cradle extending between and connecting the first support arm and the second support arm, wherein the cradle is sized and shaped for snap-fit engagement with the handle of the intraluminal imaging device. The base component may be press fit engaged with the engagement component. The engagement component may extend across the base component in a direction perpendicular to a length of the base component. In some aspects, the base component and the engagement component each have a rectangular outer profile when viewed from above such that the base component and the engagement component form an X or cross shape when press fit together and viewed from above. In some aspects, each of the first support arm and the second support arm extend at an oblique angle with respect to a base plate of the engagement component. The first and second support arms may be sized and shaped to maintain the handle of the intraluminal imaging device in a raised position spaced from a surface on which the intraluminal imaging device stabilizer is positioned when the handle of the intraluminal imaging device is snap-fit engaged with the cradle.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intra-cardiac echocardiography (ICE) imaging system according to aspects of the present disclosure.

FIG. 2 is a perspective view of an intraluminal imaging device coupled with an intraluminal imaging device stabilizer according to aspects of the present disclosure.

FIG. 3 is a top view of an intraluminal imaging device coupled with an intraluminal imaging device stabilizer according to aspects of the present disclosure.

FIG. 4 is a side view of an intraluminal imaging device coupled with an intraluminal imaging device stabilizer according to aspects of the present disclosure.

FIG. 5 is an end view of an intraluminal imaging device coupled with an intraluminal imaging device stabilizer according to aspects of the present disclosure.

FIG. 6 is a perspective view of an intraluminal imaging device stabilizer according to aspects of the present disclosure.

FIG. 7 is a perspective view of an engagement component of an intraluminal imaging device stabilizer according to aspects of the present disclosure.

FIG. 8 is a side view of an engagement component of an intraluminal imaging device stabilizer according to aspects of the present disclosure.

FIG. 9 is a perspective view of a base component of an intraluminal imaging device stabilizer according to aspects of the present disclosure.

FIG. 10 is a side view of a base component of an intraluminal imaging device stabilizer according to aspects of the present disclosure.

FIG. 11 is a flow diagram of an intraluminal imaging workflow according to aspects of the present disclosure.

FIG. 12 is a top view showing an intraluminal imaging device stabilizer being used with intraluminal imaging device during an intraluminal imaging procedure according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the ICE system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

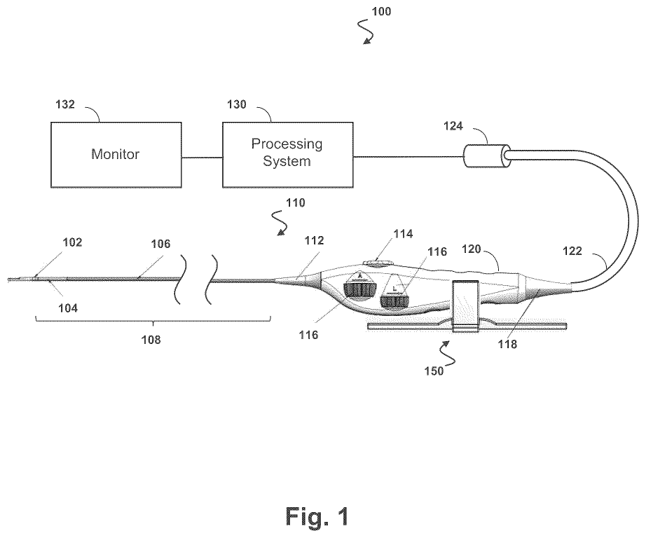

FIG. 1 is a schematic diagram of an intra-cardiac echocardiography (ICE) imaging system 100 according to aspects of the present disclosure. The system 100 may include an ICE device 110 (e.g., an ICE catheter and/or other intraluminal imaging device), a connector 124, a control and processing system 130, such as a console and/or a computer, and a monitor 132. The ICE device 110 may include a tip assembly 102, a flexible elongate member 108, and a handle 120. The flexible elongate member 108 may include a distal portion 104 and a proximal portion 106. The distal end of the distal portion 104 may be attached to the tip assembly 102. The proximal end of the proximal portion 106 may be attached to the handle 120. For example, in some instances a resilient strain relief 112 couples the proximal portion 106 to the handle 120. The handle 120 may be used for manipulation of the ICE device 110 and/or manual control of the ICE device 110. The handle 120 can include actuators 116, a clutch 114, and other steering control components for steering the ICE device 110, such as deflecting the tip assembly 102 and the distal portion 104. In some aspects, the ICE device 110 may include steering and/or control mechanisms similar to those described in U.S. Pat. No. 11,464,481, which is hereby incorporated by reference in its entirety. The tip assembly 102 may include an imaging array, imaging core, and/or imaging sensor with a plurality of ultrasound transducer elements and associated circuitry.

The handle 120 may be connected to the connector 124 via a strain relief 118 and an electrical cable 122. The connector 124 may be configured in any suitable configurations (including wired and/or wireless communications) to interconnect with the processing system 130 and the monitor 132 for processing, storing, analyzing, manipulating, and displaying data obtained from signals generated by the imaging core at the tip assembly 102. The processing system 130 can include one or more processors, memory, one or more input devices, such as keyboards and any suitable command control interface device. The processing system 130 can be operable to facilitate the features of the ICE imaging system 100 described herein. For example, the processor can execute computer readable instructions stored on the non-transitory tangible computer readable medium. The monitor 132 can be any suitable display device, such as liquid-crystal display (LCD) panel or the like.

In operation, a physician or a clinician advances the flexible elongate member 108 into a vessel within a heart anatomy. The tip assembly 102 and the flexible elongate member 108 may be shaped and sized for insertion into vessels of a patient body. The flexible elongate member 108 may be composed of any suitable material, such as Pebax® polyether block amides. The distal portion 104 and the proximal portion 106 may be tubular in shape and may include a primary lumen and one or more pullwire lumens extending longitudinally along the flexible elongate member 108. The primary lumen may be sized and shaped to accommodate an electrical cable interconnecting the tip assembly 102 and the connector 124 for transferring data (e.g., echo signals) obtained from the transducer elements. In some embodiments, the primary lumen can be sized and shaped to accommodate other components for diagnostic and/or therapy procedures. The pullwire lumens may be sized and shaped to accommodate pullwires, for example, extending from the distal portion 104 to the handle 120. The pullwires may be coupled to the actuators 116 and/or the clutch 114 such that the flexible elongate member 108 and the tip assembly 102 are deflectable based on actuations of the actuators 116 and/or the clutch 114. In some instances, the primary lumen may be sized and shaped to facilitate alignment of the pullwire lumens. In addition, the tubular body of the flexible elongate member 108 may include a lined variable braided reinforcement layer configured to provide flexibility and kink resistance.

Dimensions of the flexible elongate member 108 can vary in different embodiments. Generally, the flexible elongate member 108 may be positioned within any lumen or area within a patient body. In some instances, the flexible elongate member 108 may be sized and/or shaped for positioning with one or more particular lumens and/or target areas with the patient body. In some aspects, the flexible elongate member 108 can be a catheter having an outer diameter between about 8 and about 12 French (Fr) and can have a total length between about 80 centimeters (cm) to about 120 cm, where the proximal portion 106 can have a length between about 70 cm to about 118 cm and the distal portion 104 can have a length between about 2 cm to about 10 cm. While aspects described herein may refer to the ICE device 110, the concepts of the present disclosure may be applied to other types of intraluminal imaging devices, including IVUS, OCT, and/or other imaging modalities.

The physician or clinician can steer the flexible elongate member 108 to a desired position within the patient body. In this regard, the desired position may be near an area of interest to be imaged by the ICE device 110. In some aspects, the physician or clinician steers the flexible elongate member 108 by controlling the actuators 116 and the clutch 114 on the handle 120. For example, one actuator 116 may deflect the tip assembly 102 and the distal portion 104 in a left-right plane and the other actuator 116 may deflect the tip assembly 102 and the distal portion 104 in an anterior-posterior plane. The clutch 114 may provide a locking mechanism to lock the positions of the actuators 116 and, in turn, the deflection of the flexible elongate member 108 while imaging the area of interest.

The system 100 includes an intraluminal imaging device stabilizer 150. As discussed in greater detail below, the stabilizer 150 may facilitate improved imaging workflows. In this regard, the stabilizer 150 may be utilized to maintain the tip assembly 102 in a desired imaging location without requiring the operator or another person to continue holding the handle 120 during the procedure. The stabilizer 150 may be configured for snap-fit engagement with the handle 120 of the ICE device 110 such that undesirable movement of the tip assembly 102 inside the patient is avoided. Use of the stabilizer 150 with the ICE device 110 can allow an assistant that might otherwise be required to hold the handle 120 to maintain the position of the tip assembly 102 to handle other tasks and/or responsibilities associated with the procedure. In some aspects, the stabilizer 150 may include a raised support structure sized and shaped for snap-fit engagement with the handle 120 of the ICE device 110. The raised support structure may be configured to maintain the handle of the ICE device 110 in a raised position spaced from a surface (e.g., operating table, bed, patient, etc.) on which the stabilizer 150 is positioned. The intraluminal imaging device stabilizer 150 may be a plastic disposable sterilized holder packaged and/or shipped in two pieces. The intraluminal imaging device stabilizer 150 may be used on the procedure table to secure and enable controlled clocking/rotational motions of the intraluminal imaging device and/or secure the handle within the sterile packaging.

The intraluminal imaging device stabilizer 150 can ensure that the handle 120 of the intraluminal imaging device will be maintained in position when the catheter has been navigated to the target and/or enables rotation of distal portion of the intraluminal imaging device in the target area, avoiding the need for an additional operator to hold the catheter or use other ad hoc devices/techniques that may not avoid movement after the intraluminal imaging device has been positioned. This intraluminal imaging device stabilizer 150 may save procedure time by not having to renavigate when a position is lost and saves personnel cost because most procedures are targeting a single operator to complete the procedure. The intraluminal imaging device stabilizer 150 also provides peace of mind to the operator because they know that they can confidently rely on function of the intraluminal imaging device stabilizer 150, creating less distraction and leading to better results.

The imaging process may include activating the ultrasound transducer elements of the tip assembly 102 to produce ultrasonic energy. In some instances, one or more aspects of the imaging process may occur while the handle 120 of the ICE device 110 is engaged with the stabilizer 150 and/or while the handle 120 is not engaged with the stabilizer 150. A portion of the ultrasonic energy may be reflected by the area of interest and the surrounding anatomy. The ultrasound echo signals may be received by the ultrasound transducer elements. The connector 124 may transfer the received echo signals to the processing system 130. The processing system 130 may process the received echo signals to generate the ultrasound image(s) and output the image(s) to the monitor 132 for display. In some aspects, the processing system 130 may control the activation of the ultrasound transducer elements and/or the repletion of the echo signals. In some embodiments, the processing system 130 and the monitor 132 may be part of the same system.

The system 100 may be utilized in a variety of applications such as transseptal lumen punctures, left atrial appendage closures, atrial fibrillation ablation, and valve repairs and can be used to image vessels and structures within a patient body. Although the system 100 is described in the context of ICE catheterization procedures, the system 100 is suitable for use with any catheterization procedure, including structural heart, cardiac, peripheral, and/or otherwise. In addition, the tip assembly 102 may include any suitable physiological sensor, component, and/or functional element for diagnosis, treatment, and/or therapy, such as pressure sensor(s), flow sensor(s), force sensor(s), doppler sensor(s), etc. The physiological sensors may be provided in addition to and/or in lieu of the imaging element(s). Thus, the handle 120 can be used to guide articulation and/or positioning of any type of functional element included in the distal portion 104 of the ICE device 110.

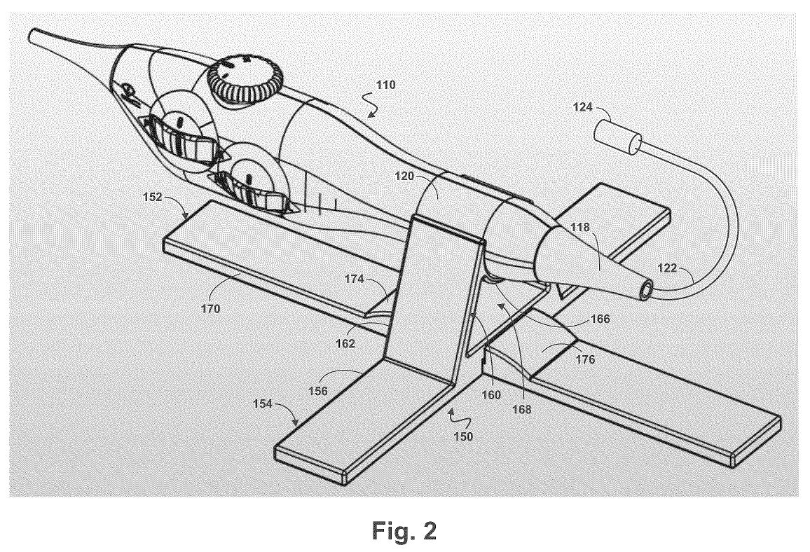

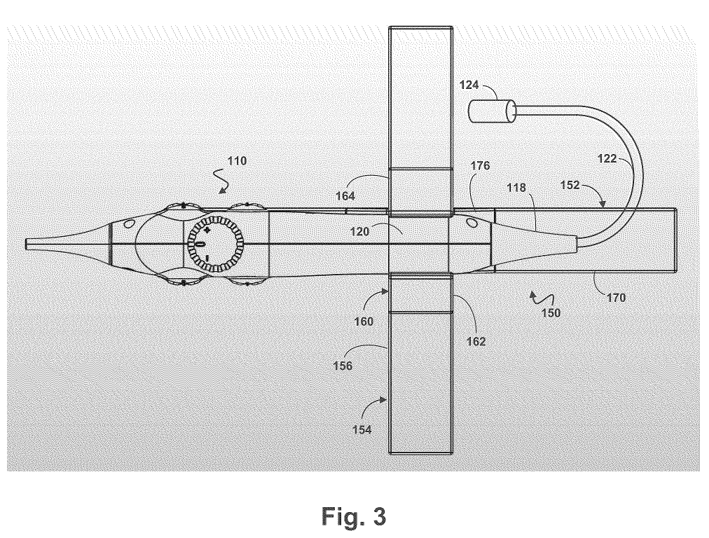

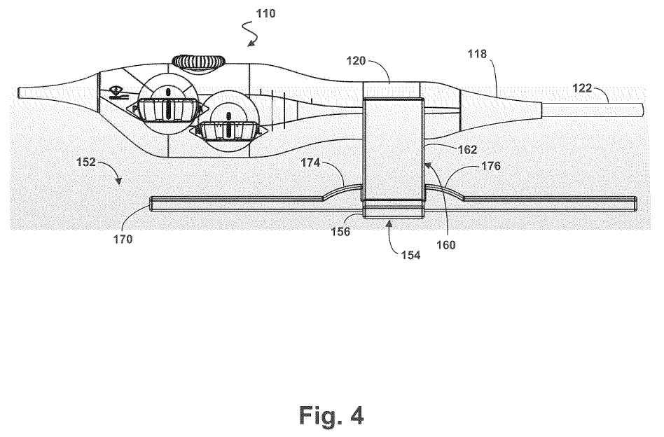

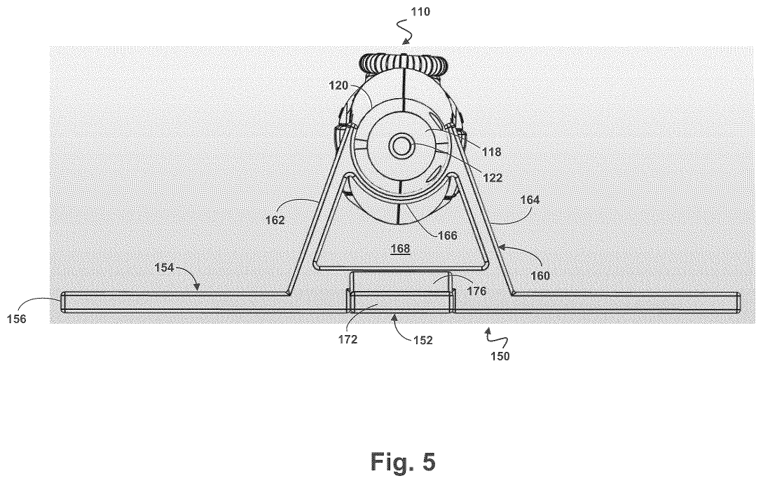

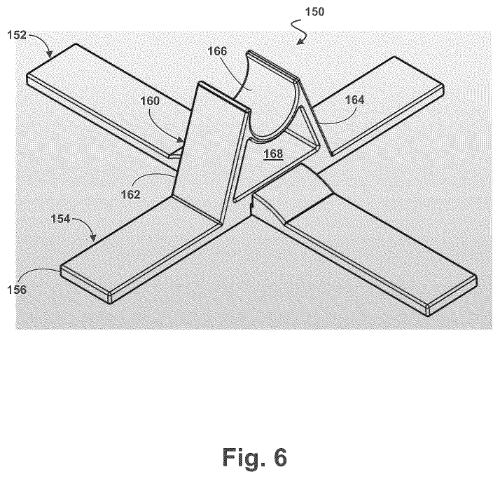

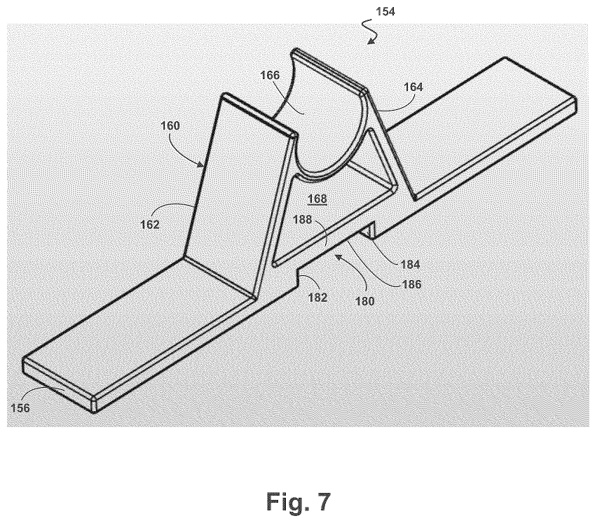

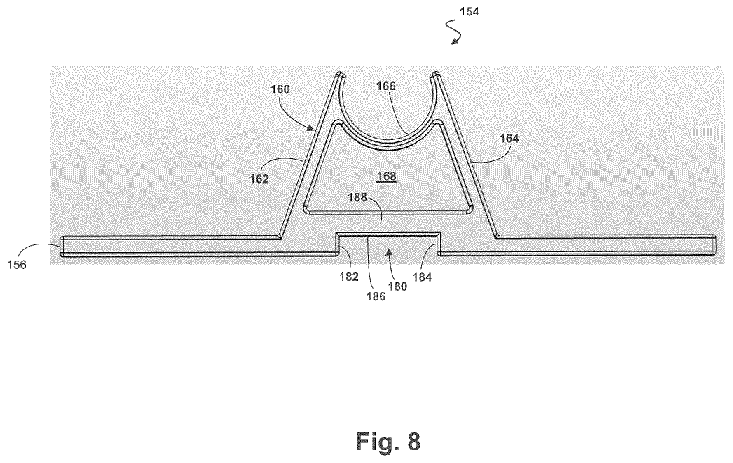

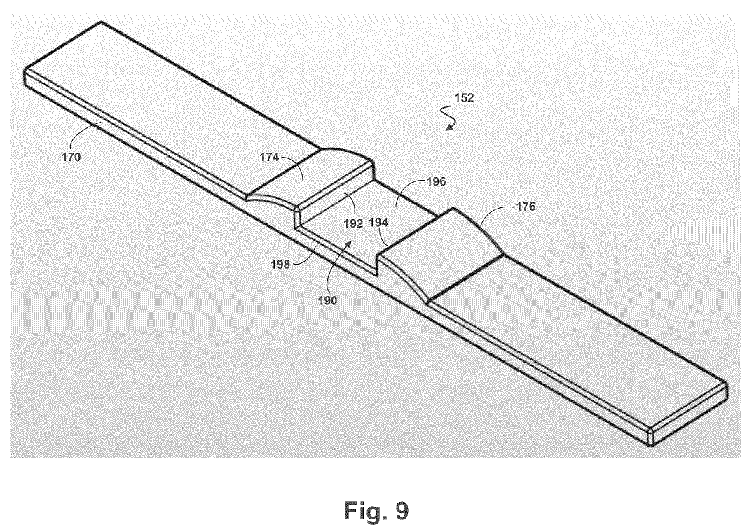

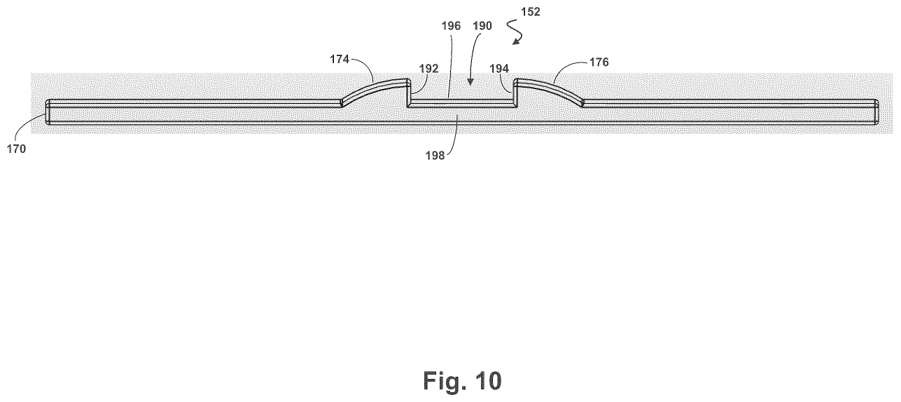

Referring to FIGS. 2-10 additional details regarding an intraluminal imaging system, including the intraluminal imaging device stabilizer 150, will be described. In this regard, FIG. 2 is a perspective view of an intraluminal imaging device (e.g., ICE device 110) coupled with the intraluminal imaging device stabilizer 150 according to aspects of the present disclosure; FIG. 3 is a top view of the intraluminal imaging device coupled with the intraluminal imaging device stabilizer 150 according to aspects of the present disclosure; FIG. 4 is a side view of the intraluminal imaging device coupled with the intraluminal imaging device stabilizer 150 according to aspects of the present disclosure; FIG. 5 is an end view of the intraluminal imaging device coupled with the intraluminal imaging device stabilizer 150 according to aspects of the present disclosure; FIG. 6 is a perspective view of the intraluminal imaging device stabilizer 150 according to aspects of the present disclosure; FIG. 7 is a perspective view of an engagement component 154 of the intraluminal imaging device stabilizer 150 according to aspects of the present disclosure; FIG. 8 is a side view of the engagement component 154 of the intraluminal imaging device stabilizer 150 according to aspects of the present disclosure; FIG. 9 is a perspective view of a base component 152 of the intraluminal imaging device stabilizer 150 according to aspects of the present disclosure; and FIG. 10 is a side view of the base component 152 of the intraluminal imaging device stabilizer 150 according to aspects of the present disclosure.

As shown in FIGS. 1-5, in some aspects an intraluminal imaging system may comprise an intraluminal imaging device (e.g., an ICE device, an IVUS device, an OCT device, or other suitable intraluminal imaging device). The intraluminal imaging device may include a handle 120 sized and shaped for handheld use. The intraluminal imaging device may include a distal portion coupled to the handle 120. The distal portion may be sized and shaped for introduction into a patient body and including an imaging core (e.g., a single imaging element, an array of imaging elements, an ultrasound transducer, an array of ultrasound transducers, an OCT element, an array of OCT elements, or otherwise). The intraluminal imaging system may also comprise an intraluminal imaging device stabilizer 150. In some instances, the intraluminal imaging device and the intraluminal imaging device stabilizer 150 may be provided together in the same package. In some instances, the intraluminal imaging device stabilizer 150 may be provide structural stability within the box, including potentially supporting and/or protecting aspects of the intraluminal imaging device. In other instances, the intraluminal imaging device and the intraluminal imaging device stabilizer 150 may be packaged separately. In some instances, the intraluminal imaging device stabilizer 150 is a multi-piece structure that is packaged separately and then assembled. For example, the intraluminal imaging device stabilizer 150 may include a base component 152 and an engagement component 154. The base component 152 and the engagement component 154 may be made of any suitable material(s), including without limitation plastics, rubbers, metals, and/or combinations thereof. In some aspects, the material hay have shore hardness (or durometer) between about 70-90 (scale D) and/or a flexural strength around 70-110 MPa. In this regard, the material or combination of materials may be selected such that the intraluminal imaging device stabilizer 150 flexes open as the user snaps the handle of the intraluminal imaging device into the stabilizer and flexes back closed to securely hold onto the handle effectively once engaged. Further, the material or combination of materials may be selected such that intraluminal imaging device stabilizer is not too brittle (which could lead to cracking and/or breaking during use) but also not too flexible (which could lead to not holding the handle in a fixed position).

In some instances, the base component 152 and the engagement component 154 are separate components that are coupled together for use. For example, in some instances the base component 152 may be press fit engaged with the engagement component 154 (sec, e.g., FIGS. 2-6). The engagement component 154 may extend across the base component 152 in a direction perpendicular to a length of the base component (see, e.g., FIGS. 2-6). In some instances, the base component 152 and the engagement component 154 each have a rectangular outer profile when viewed from above such that the base component 152 and the engagement component 154 form an X or cross shape when press fit together and viewed from above (see, e.g., FIGS. 2, 3, and 6).

As best shown in FIGS. 5-8, the engagement component 154 may include a base plate 156. As illustrated, in some examples the base plate 156 may have a rectangular outer profile defined by a length and a width. In some instances, the length of the base plate 156 may be between about 10 cm and about 30 cm, including 15 cm, 20 cm, 25 cm, or other suitable length. In some instances, the width of the base plate 156 may be between about 3 cm and about 6 cm, including 3 cm, 4 cm, 5 cm, or other suitable width. In some instances, the width of the base plate 156 is equal or approximately equal to a cylindrical portion of the handle 120 of the intraluminal imaging device 120 that the engagement component 154 is configured to engage with. In some instances, the base plate 156 has thickness between about 0.1 cm and about 1.0 cm, including 0.4 cm, 0.6 cm 0.8 cm, or other suitable thickness. In other instances, the base plate 156 may have other geometrical and/or non-geometrical profiles, including combinations thereof.

In some instances, the base component 152 may include a base plate 170. The base plate 170 may have a rectangular outer profile defined by a length and a width. In some instances, the length of the base plate 170 may be between about 10 cm and about 30 cm, including 15 cm, 20 cm, 25 cm, or other suitable length. In some instances, the width of the base plate 170 may be between about 3 cm and about 6 cm, including 3 cm, 4 cm, 5 cm, or other suitable width. In some instances, the base plate 170 has thickness between about 0.1 cm and about 1.0 cm, including 0.4 cm, 0.6 cm 0.8 cm, or other suitable thickness. In other instances, the base plate 170 may have other geometrical and/or non-geometrical profiles, including combinations thereof. In some instances, the base plate 170 of the base component 152 has an outer profile and/or thickness the same as or similar to the base plate 156 of the engagement component 154.

The engagement component 154 may also include a raised support structure 160 sized and shaped for snap-fit engagement with the handle 120 of the intraluminal imaging device. As best seen in FIGS. 5-8, in some aspects the raised support structure 160 of the engagement component 154 includes a first support arm 162, a second support arm 164, and a cradle 166 extending between and connecting the first support arm 162 and the second support arm 164. In some instances, each of the first support arm 162 and the second support arm 164 extend at an oblique angle (e.g., 15-75 degrees or other suitable angle) with respect to a base plate 156 of the engagement component 154. An opening 168 may be defined between the first support arm, the second support arm 164, and the cradle 166. The cradle 166 may be sized and shaped for snap-fit engagement with the handle 120 of the intraluminal imaging device (e.g., the ICE device 110). For example, the cradle 166 may be configured to provide an annular snap-fit with the handle 120 (e.g., as shown in FIGS. 1-5) where a hoop-strain may be utilized to hold the handle in place. In this regard, the circumference of the opening defined by the first support arm 162, the second support arm 164, and the cradle 166 may expand as the handle 120 is pushed between the first support arm 162 and the second support arm 164 and into the cradle 166. In this regard, the first support arm 162 and the second support arm 164 of the engagement component 154 may expand or deflect outward and then snap-fit around the handle once the handle is fully seated within the cradle 166.

The size, shape, and/or material(s) of the first support arm 162, the second support arm 164, and the cradle 166 are configured for snap-fit engagement with the handle of the intraluminal imaging device. As shown, the cradle 166 may have a generally cylindrical shape with an opening at the top for receiving the handle 120. In this regard, as seen in FIG. 8, the cradle 166 may extend circumferentially between about 225 degrees and about 235 degrees such that the opening for receiving the handle 120 is between about 125 degrees and about 135 degrees. In some instances, each of the first support arm 162, the second support arm 164, and the cradle 166 may have a thickness between about 0.1 cm and about 1.0 cm, including 0.2 cm, 0.25 cm, 0.3 cm, 0.4 cm, 0.5 cm, 0.6 cm, 0.75 cm, 0.8 cm, or other suitable thickness. In some implementations, the first support arm 162 and the second support arm 164 each have a thickness about 0.5 cm and the cradle 166 has a thickness of about 0.3 cm. Further, the first support arm 162, the second support arm 164, and the cradle 166 may be formed of the same and/or different material(s) than the base plate 156 of the engagement component 154. In some instances, the raised support structure 160 may be configured to provide snap-fit engagement with the handle 120 using a cantilevered and/or a torsional snap-fit arrangement.

Further, in some instances, the first support arm 162, the second support arm 164, and/or the cradle 166 are each sized and shaped to maintain the handle 120 of the intraluminal imaging device (e.g., ICE device 110) in a raised position spaced from a surface on which the intraluminal imaging device stabilizer 150 is positioned. For example, each of the first support arm 162 and the second support arm 164 may extend a sufficient distance from the base plate 156 such that the intraluminal imaging device is spaced from the base plate 156 when the handle of the intraluminal imaging device is engaged with the raised support structure 160. In some instances, as shown in FIGS. 2, 4, and 5, the first support arm 162, the second support arm 164, and/or the cradle 166 engage the handle 120 of the intraluminal imaging device such that the intraluminal imaging device is maintained above the base plate 156, as well as any surface on which the base plate may be positioned (e.g., operating table, bed, patient, etc.). Further, in some instances the first support arm 162, the second support arm 164, and/or the cradle 166 are configured to maintain the intraluminal imaging device in a position generally parallel to the base plate 156 of the engagement component 154 and/or generally parallel to the base component 152.

In some instances, engagement of the handle 120 with the intraluminal imaging device stabilizer 150 maintains the position of the distal portion of the intraluminal imaging device within the patient. In some instances, the distal portion of the intraluminal imaging device may be adjusted while the handle 120 of the intraluminal imaging device is snap-fit engaged with the intraluminal imaging device stabilizer 150. For example, in some instances one or more actuators of the handle 120 may be used to adjust an orientation of an imaging tip and/or imaging core of the intraluminal imaging device while snap-fit engaged with the intraluminal imaging device stabilizer 150. Further, the location of the distal portion of intraluminal imaging device, including the imaging tip and/or imaging core, may be adjusted through movement of the handle 120 (e.g., translation, rotation, pivoting, other movements, and/or combinations thereof) while snap-fit engaged with the intraluminal imaging device stabilizer. In some instances, the snap-fit engagement of the handle 120 with the intraluminal imaging device stabilizer allows the handle 120 to be rotated relative to the intraluminal imaging device stabilizer 150 through use of sufficient rotational force by a user but provides sufficient frictional engagement to maintain the handle 120 in a fixed position once the user stops rotating the handle. In some instances, the handle 120 may be rotated any suitable amount (e.g., between 0-360 degrees) and maintained at the desired orientation once the user stops the rotation. In this manner, adjustments to the rotational position of the handle 120 relative to the intraluminal imaging device stabilizer 150 may be made without completely separating or disengaging the handle 120 from the intraluminal imaging device stabilizer 150. For example, the size, shape, and/or material(s) of the first support arm 162, the second support arm 164, and the cradle 166 may be selected based on the size, shape, and material(s) of the handle 120 to allow the handle 120 to be rotated relative to raised support structure 160 by rotational force by a user but provides sufficient frictional engagement to maintain the handle 120 in a fixed position once the user stops rotating the handle. In this manner, the intraluminal imaging device stabilizer 150 may help a physician stabilize the image(s) obtained by securing the handle of the intraluminal imaging device to the raised support structure, while still allowing purposeful rotational or translational changes to be made.

The base component 152 and the engagement component 154 may each include one or more structural features to facilitate the press-fit engagement of the two components. For example, as shown in FIGS. 7 and 8, in some instances the base plate 156 of the engagement component 154 may include a recess 180 sized and shaped to engage with a mating structure (e.g., a recess, a projection, and/or combination(s) thereof) of the base component 152. The recess 180 may be defined by a first lateral bounding surface 182, a second lateral bounding surface 184, and an upper bounding surface 186. As shown in FIGS. 9 and 10, in some instances the base component 152 may include a raised portion 174 and raised portion 176 separated by a recess 190 sized and shaped to engage with a mating structure (e.g., a recess, a projection, and/or combination(s) thereof) of the engagement component 154. The recess 190 may be defined by a first lateral bounding surface 192, a second lateral bounding surface 194, and a lower bounding surface 196. In some instances, the lower bounding surface 196 of the base component 152 may engage with the upper bounding surface 186 of the engagement component 154 when the base component 152 and the engagement component 154 are press fit together. Further, the first lateral bounding surface 182 and the second lateral bounding surface 184 of the engagement component 154 may engage opposing sides of the base component 152 when the base component 152 and the engagement component 154 are press fit together. Similarly, the first lateral bounding surface 192 and the second lateral bounding surface 194 of the base component 152 may engage opposing sides of the engagement component 154 when the base component 152 and the engagement component 154 are press fit together. In some instances, the bottom surfaces of the base component 152 and the engagement component 154 are coplanar (or parallel) when the base component 152 and the engagement component 154 are press fit together.

It is understood that the base component 152 and the engagement component 154 may be coupled together in other manners, including without limitation snap fitting, coupling with one or more fasteners (e.g., bolts, nuts, washers, screws, etc.), and/or otherwise connecting the base component 152 and the engagement component 154 together.

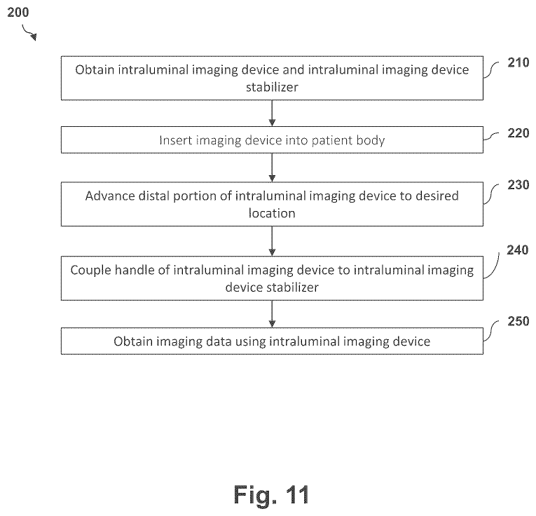

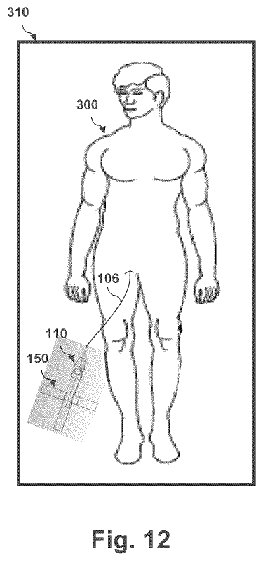

Referring to FIGS. 11-12 additional details regarding a workflow associated with using an intraluminal imaging device stabilizer with an intraluminal imaging device during an intraluminal imaging procedure will be described. In this regard, FIG. 11 is a flow diagram of an intraluminal imaging workflow according to aspects of the present disclosure; and FIG. 12 is a top view showing an intravascular imaging device handle stabilizer 150 being used with a patient on an operating table during an intraluminal imaging workflow according to aspects of the present disclosure.

FIG. 11 is a flow diagram of method 200 of obtaining imaging data from within a patient body using an intraluminal imaging device. It is understood that the actions of method 200 may be performed in a different order than shown in FIG. 11, additional actions can be provided before, during, and after the described actions, and/or some of the described actions can be replaced and/or eliminated. Some of the actions of the method 200 can be carried out by a user of the intraluminal imaging device.

At action 210, the method 200 includes obtaining an intraluminal imaging device and an intraluminal imaging device stabilizer. The intraluminal imaging device may be an ICE device (e.g., similar to or the same as ICE device 110 described above in the context of FIGS. 1-5), an IVUS device, an OCT device, or other suitable imaging device configured for introduction into a patient. The intraluminal imaging device may include a flexible elongate member extending from a handle. A distal portion of the flexible elongate member may include an imaging core. The handle may control one or more pullwire segments extending along a length of the flexible elongate member to facilitate selective deflection of the distal portion, including the imaging core.

The intraluminal imaging device stabilizer may be similar to or the same as the intraluminal imaging device stabilizer 150 described above in the context of FIGS. 1-10. In some instances, the intraluminal imaging device and the intraluminal imaging device stabilizer 150 may be provided together in the same package. In other instances, the intraluminal imaging device and the intraluminal imaging device stabilizer 150 may be packaged separately. In some instances, the intraluminal imaging device stabilizer 150 is a multi-piece structure that is packaged separately and then assembled. For example, in some instance the method 200 includes assembling the intraluminal imaging device stabilizer 150. In some aspects, assembling the intraluminal imaging device stabilizer 150 may include coupling a base component and an engagement component. Coupling the base component and the engagement component may include press fitting, snap fitting, coupling with one or more fasteners (e.g., bolts, nuts, washers, screws, etc.), and/or otherwise connecting the base component and the engagement component together. The engagement component may extend across the base component in a direction perpendicular to a length of the base component. In some instances, the base component and the engagement component each have a rectangular outer profile when viewed from above (see, e.g., FIG. 3) such that the base component and the engagement component form an X or cross shape when press fit together and viewed from above (see, e.g., FIGS. 2, 3, 6, and 12).

In some instances, the intraluminal imaging device stabilizer 150 includes a raised support structure. The raised support structure may be sized and shaped for snap-fit engagement with a handle of the intraluminal imaging device. Further, the raised support structure may be sized and shaped to maintain the handle of the intraluminal imaging device in a raised position spaced from a surface (e.g., operating table, bed, patient, etc.) on which the intraluminal imaging device stabilizer 150 is positioned. In some aspects, the engagement component may include the raised support structure. In some instances, the raised support structure includes a first support arm, a second support arm, and a cradle extending between and connecting the first support arm and the second support arm. The cradle may be sized and shaped for snap-fit engagement with the handle of the intraluminal imaging device. In some instances, each of the first support arm and the second support arm extend at an oblique angle (e.g., between ˜10-80 degrees in some instances) with respect to a base plate of the engagement component. In this regard, the first and second support arms may be sized and shaped to maintain the handle of the intraluminal imaging device in the raised position spaced from a surface on which the intraluminal imaging device stabilizer is positioned when the handle is snap-fit engaged with the cradle.

At action 220, the method 200 includes inserting the intraluminal imaging device into a patient body. For example, a distal portion of the intraluminal imaging device can be positioned within any suitable lumen with the body of a patient. In some instances, the distal portion of the intraluminal imaging device is advanced through the femoral or jugular artery when accessing the anatomy of the patient and steered to the heart to acquire images for associated medical procedures. For example, as shown in FIG. 12, a distal portion 104 of an ICE device 110 having an imaging core or assembly may be inserted into a femoral artery of a patient 300 on an operating table 310.

At action 230, the method 200 includes advancing a distal portion to the intraluminal imaging device to a desired location within the patient. Advancing the distal portion may include steering the distal portion of the intraluminal imaging device using one or more steering mechanisms of the handle. One or more components of a steering mechanism can include pulley(s) coupled to the pullwire segment(s), axle(s), and/or actuation control member(s). The actuation control members can be coupled to the pullwire segments via the pulleys such that movement of the actuation control members causes corresponding deflection of the distal portion of the flexible elongate member. A clutch mechanism may include a clutch control member, a clutch cam, a clutch spring, and frictional members. The clutch control member can be moved to increase or decrease the compression force on the clutch cam. The clutch cam in turn applies the suitable compression force on the clutch spring. The frictional members are positioned adjacent to and/or in contact with the actuation control members. The actuation control members may be urged into contact with the frictional members in response to the control force. Increased contact slows down the rate of return to the non-deflected state. Decreased contact speeds up the rate of return to the non-deflected state. In this manner, the method 200 may include controlling the rate at which the distal portion of the intraluminal imaging device returns to a non-deflected state using the clutch mechanism.

At action 240, the method 200 includes coupling the handle of the intraluminal imaging device to the intraluminal imaging device stabilizer. In some aspects, the handle is snap-fit engaged with an engagement component of the intraluminal imaging device stabilizer. For example, the handle may be snap-fit engaged with the engagement component using an annular snap-fit (e.g., as shown in FIGS. 1-5) where a hoop-strain may be utilized to hold the handle in place. In this regard, the circumference of the engagement component of the intraluminal imaging device stabilizer may expand as the handle is pushed into an opening of the engagement component and then snap-fit around the handle once the handle is fully seated within the opening. The size, shape, and/or material(s) of the engagement component are configured for snap-fit engagement with the handle of the intraluminal imaging device. In other instances, the handle may be snap-fit engaged with the engagement component using a cantilevered and/or a torsional snap-fit arrangement.

In some instances, the handle may be snap-fit engaged with a raised support structure that maintains the handle of the intraluminal imaging device in a raised position spaced from a surface (e.g., operating table, bed, patient, etc.) on which the intraluminal imaging device stabilizer 150 may be positioned. As shown in FIG. 12, the intraluminal imaging device stabilizer 150 may be placed on an operating table 310 next to the patient 300 and maintain the handle 120 in an elevated position relative to the operating table. In some instances, the raised support structure includes a first support arm, a second support arm, and a cradle extending between and connecting the first support arm and the second support arm, where the handle may be snap-fit engaged with the cradle. While the intraluminal imaging device stabilizer 150 may be configured for snap-fit engagement with the handle of the intraluminal imaging device, in some instances the intraluminal imaging device stabilizer 150 may be configured (e.g., sized and shaped and/or made of suitable material(s)) to provide an interference fit, a mount, or otherwise hold the handle of the intraluminal imaging device in a fixed position.

At action 250, the method 200 includes obtaining imaging data using the intraluminal imaging device from within the patient body. In some instances, the imaging data is obtained while the intraluminal imaging device is snap-fit engaged with the intraluminal imaging device stabilizer. Further, in some instances, the positioning of the distal portion of the intraluminal imaging device within the patient may be changed and additional imaging data obtained. In this regard, the distal portion of the intraluminal imaging device may be adjusted while the handle of the intraluminal imaging device is snap-fit engaged with the intraluminal imaging device stabilizer. For example, in some instances one or more actuators of the handle may be used to adjust an orientation of an imaging tip and/or imaging core of the intraluminal imaging device while snap-fit engaged with the intraluminal imaging device stabilizer. Further, the location of the distal portion of intraluminal imaging device, including the imaging tip and/or imaging core, may be adjusted through movement of the handle (e.g., translation, rotation, pivoting, other movements, and/or combinations thereof) while snap-fit engaged with the intraluminal imaging device stabilizer. In some instances, the snap-fit engagement of the handle with the intraluminal imaging device stabilizer allows the handle to be rotated relative to the intraluminal imaging device stabilizer through use of sufficient rotational force by a user but provides sufficient frictional engagement to maintain the handle in a fixed position once the user stops rotating the handle. In this manner, adjustments to the rotational position of the handle relative to the intraluminal imaging device stabilizer may be made without completely separating or disengaging the handle from the intraluminal imaging device stabilizer.

In other instances, the handle of the intraluminal imaging device may be disengaged from the intraluminal imaging device stabilizer during repositioning of the distal portion of the intraluminal imaging device. Once the distal portion is in the new desired position, then the handle may be reengaged (e.g., via snap-fit engagement) with the intraluminal imaging device stabilizer. Imaging data may be obtained at each of plurality of locations and/or orientations of the distal portion of the intraluminal imaging device. The method 200 can further include actions related to processing, storing, displaying, manipulating, and/or reviewing the obtained imaging data, including making medical treatment decisions and executing the associated treatments.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A stabilizer for an intraluminal imaging device, the stabilizer comprising:
a base component; and
an engagement component coupled to the base component, the engagement component including a raised support structure sized and shaped for snap-fit engagement with a handle of an intraluminal imaging device, wherein the raised support structure comprises:
a first support arm;
a second support arm; and
a cradle extending between and connecting the first support arm and the second support arm, wherein the cradle is sized and shaped for snap-fit engagement with the handle of the intraluminal imaging device.

2. The stabilizer of claim 1, wherein the base component is press fit engaged with the engagement component.

3. The stabilizer of claim 2, wherein the engagement component extends across the base component in a direction perpendicular to a length of the base component.

4. The stabilizer of claim 2, wherein the base component and the engagement component each have a rectangular outer profile when viewed from above such that the base component and the engagement component form an X shape or a cross shape when press fit together and viewed from above.

5. The stabilizer of claim 1, wherein each of the first support arm and the second support arm extend at an oblique angle with respect to a base plate of the engagement component.

6. The stabilizer of claim 5, wherein the first support arm and the second support arm are sized and shaped to maintain the handle of the intraluminal imaging device in a raised position spaced from a surface on which the stabilizer is positioned.

7. An intraluminal imaging system, comprising:
an intraluminal imaging device including:
a handle sized and shaped for handheld use; and
a distal portion coupled to the handle, the distal portion sized and shaped for introduction into a patient body and including an imaging array; and
an intraluminal imaging device stabilizer including:
a base component; and
an engagement component coupled to the base component, the engagement component including a raised support structure sized and shaped for snap-fit engagement with the handle of the intraluminal imaging device.

8. The system of claim 7, wherein the base component is press fit engaged with the engagement component.

9. The system of claim 8, wherein the engagement component extends across the base component in a direction perpendicular to a length of the base component.

10. The system of claim 8, wherein the base component and the engagement component each have a rectangular outer profile when viewed from above such that the base component and the engagement component form an X shape or a cross shape when press fit together and viewed from above.

11. The system of claim 7, wherein the raised support structure includes:
a first support arm;
a second support arm; and
a cradle extending between and connecting the first support arm and the second support arm, wherein the cradle is sized and shaped for snap-fit engagement with the handle of the intraluminal imaging device.

12. The system of claim 11, wherein each of the first support arm and the second support arm extend at an oblique angle with respect to a base plate of the engagement component.

13. The system of claim 12, wherein the first support arm and the second support arm are sized and shaped to maintain the handle of the intraluminal imaging device in a raised position spaced from a surface on which the intraluminal imaging device stabilizer is positioned.

14. A method of intraluminal imaging, comprising:
inserting an intraluminal imaging device into a patient body;
advancing a distal portion of the intraluminal imaging device to a desired location within the patient body;
coupling a handle of the intraluminal imaging device to an intraluminal imaging device stabilizer by snap-fitting the handle to a raised support structure of the intraluminal imaging device stabilizer; and
obtaining imaging data of a region of interest of the patient body using the intraluminal imaging device.

15. The method of claim 14, further comprising:
rotating, while the handle of the intraluminal imaging device is positioned within the raised support structure of the intraluminal imaging device stabilizer, the handle of the intraluminal imaging device relative to the intraluminal imaging device stabilizer, wherein rotating the handle adjust an orientation of the distal portion of the intraluminal imaging device.

16. The method of claim 14, further comprising:
moving, while the handle of the intraluminal imaging device is coupled to the intraluminal imaging device stabilizer, the handle of the intraluminal imaging device and the intraluminal imaging device stabilizer to adjust a position of the distal portion of the intraluminal imaging device.

17. The method of claim 14, further comprising:
adjusting, while the handle of the intraluminal imaging device is coupled to the intraluminal imaging device stabilizer, an orientation of an imaging array in the distal portion of the intraluminal imaging device using at least one actuator on the handle of the intraluminal imaging device.

18. The method of claim 14, wherein the assembling the intraluminal imaging device stabilizer includes coupling a base component and an engagement component.

19. The method of claim 14, wherein the coupling the base component and the engagement component includes press fitting the base component and the engagement component together.
